(19) United States
(12) Patent Application Publication
(10) Pub. No.: US 2023/0054617 A1
(43) Pub. Date: Feb. 23, 2023

(54) USE OF INTRACARDIAC BLOOD PUMPS AS A BRIDGE TO HIGH-RISK MEDICAL PROCEDURES

(71) Applicant: ABIOMED, Inc., Danvers, MA (US)

(72) Inventors: Jerald Wayne Curran, Danvers, MA (US); Jin Kwang Kim, Danvers, MA (US)

(73) Assignee: ABIOMED, Inc., Danvers, MA (US)

(21) Appl. No.: 17/888,712

(22) Filed: Aug. 16, 2022

Related U.S. Application Data
(60) Provisional application No. 63/234,568, filed on Aug. 18, 2021.

(51) Int. Cl. A61M 60/13; A61M 60/174; A61M 60/216; A61M 60/414; A61M 60/508; A61M 60/829

(52) U.S. Cl. CPC A61M 60/13; A61M 60/174; A61M 60/216; A61M 60/414; A61M 60/508; A61M 60/829

(57) ABSTRACT

Methods of using an intracardiac blood pump in association with medical procedures. In some cases, patients may be turned down for a medical procedure based on a risk that the procedure itself may cause the patient to experience a cardiac event during and/or following the procedure. In some examples, an intracardiac blood pump may be used to support the heart before, during, and/or after the medical procedure so as to minimize such risks, and thus enable the patient to receive critical treatment that might otherwise be denied.

### CROSS-REFERENCE TO RELATED APPLICATIONS

The present application claims priority to U.S. Provisional Application No. 63/234,568 filed Aug. 18, 2021, the disclosure of which is hereby incorporated by reference herein in its entirety.

### BACKGROUND

Intracardiac blood pumps have traditionally been used to temporarily assist the pumping function of a patient's heart during emergent cardiac procedures, such as a stent placement, performed after the patient suffers a heart attack, cardiac arrest, and/or cardiogenic shock. Intracardiac blood pumps also may be used to take the load off of a patient's heart to allow the heart to recover from such a cardiac procedure or from a heart attack, cardiac arrest, cardiogenic shock, or heart damage (e.g., caused by a viral infection). In that regard, an intracardiac blood pump can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Examples of such systems include the IMPELLA® family of devices (Abiomed, Inc., Danvers Mass.).

### BRIEF SUMMARY

The present technology relates to methods of using an intracardiac blood pump as a bridge to high-risk medical procedures. For example, in some instances, a patient may require a medical procedure (e.g., a surgical procedure) but may be turned down for the procedure based on a risk that the patient may experience an adverse outcome (e.g., the procedure itself may cause the patient to experience hemodynamic instability during and/or following the procedure and/or may lead to the patient's death). In other words, the medical procedure may be considered a high-risk procedure for the patient. As described herein, high-risk medical procedures may include cardiac and non-cardiac procedures. For example, the high-risk procedure may include a gastrointestinal surgery (e.g., a cholecystectomy), a laparoscopic surgery (e.g., laparoscopic bariatric surgery), a tumor resection, an atrial fibrillation catheter ablation, a mitral valve repair, etc. In such a case, an intracardiac blood pump may be used to support the heart before, during, and/or after the procedure so as to minimize such risks, and thus enable the patient to receive critical treatment that might otherwise be denied.

In one aspect, the disclosure describes a method of administering medical treatment, comprising: identifying a patient requiring a medical procedure; making a first assessment of the patient's likelihood of experiencing one or more adverse outcomes of a set of adverse outcomes if the medical procedure were to be performed without the patient receiving support from an intracardiac blood pump before, during, or after the medical procedure; determining the patient's suitability for the medical procedure based on the first assessment; making a second assessment of the patient's likelihood of experiencing one or more adverse outcomes of the set of adverse outcomes if the medical procedure were to be performed with the patient receiving support from an intracardiac blood pump at least before, during, or after the medical procedure; determining the patient's suitability for the medical procedure based on the second assessment; and inserting the intracardiac blood pump into the patient to provide cardiac support at least before, during, or after the medical procedure. In some aspects, the method further comprises determining a period of time during which the patient would benefit from receiving support from the intracardiac blood pump, wherein the determined period of time comprises one or more of before, during, or after the medical procedure. In some aspects, inserting the intracardiac blood pump into the patient to provide cardiac support is performed for the determined period of time. In some aspects, the method further comprises performing the medical procedure on the patient. In some aspects, inserting the intracardiac blood pump into the patient is performed before, at the same time as, or after performing the medical procedure. In some aspects, the medical procedure includes a noncardiac medical procedure. In some aspects, the intracardiac blood pump is configured to provide left heart support. In some aspects, the intracardiac blood pump is configured to provide right heart support. In some aspects, the medical procedure requires the patient to be anesthetized. In some aspects, the medical procedure includes one or more of laparoscopic surgery, tumor resection, or gastrointestinal surgery. In some aspects, the medical procedure includes one or more of mitral valve repair, mitral valve replacement, ventricular tachycardia ablation or atrial fibrillation catheter ablation. In some aspects, the medical procedure includes knee or hip arthroplasty. In some aspects, the set of adverse outcomes includes one or more of hypotension, pulmonary edema, ventricular fibrillation, exacerbated ischemia, myocardial ischemia, hemodynamic collapse, cardiac arrest, stroke, heart attack, acute kidney injury, neurological decline, or death. In some aspects, the first assessment or the second assessment is based on one or more of the patient's age, height, weight, body mass index, blood pressure, cholesterol levels, liver function, kidney function, existing medical conditions, personal medical history, or family medical history. In some aspects, the first assessment or the second assessment is based on whether the patient has one or more of diabetes, an autoimmune disorder, or heart disease. In some aspects, the first assessment or the second assessment is based on statistics regarding how prevalent each adverse outcome in the set of adverse outcomes is in a given population. In some aspects, the given population comprises a group of people sharing one or more traits with the patient. In some aspects, the second assessment is based on a likelihood of the patient experiencing one or more adverse outcomes of the set of adverse outcomes as a result of implantation of the intracardiac blood pump in the patient.

### BRIEF DESCRIPTION OF DRAWINGS

FIG. 1 depicts an exemplary intracardiac blood pump assembly configured for left heart support, in accordance with aspects of the disclosure.

FIG. 2 depicts an exemplary intracardiac blood pump assembly configured for right heart support, in accordance with aspects of the disclosure.

FIG. 3 depicts an exemplary method for assessing whether a patient may benefit from treatment with an intracardiac blood pump in association with a medical procedure.

FIG. 4 depicts an exemplary method for treating a patient using an intracardiac blood pump in association with a medical procedure.

### DETAILED DESCRIPTION

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to employ the present disclosure in other suitable structures.

To provide an overall understanding of the systems, methods, and devices described herein, certain illustrative examples will be described. Although various examples may describe specific medical procedures and/or uses of intracardiac blood pumps, it will be understood that the present technology may be employed in any suitable context.

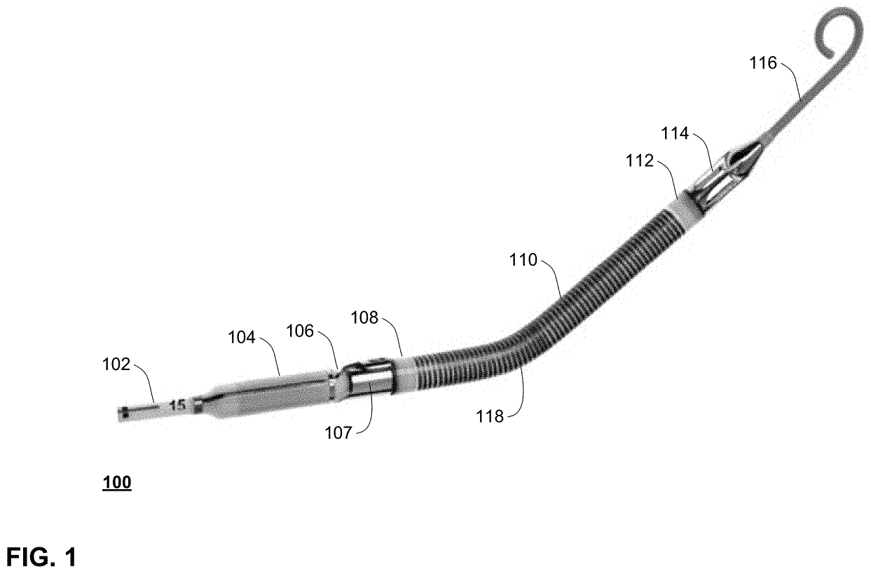

FIG. 1 depicts an exemplary intracardiac blood pump assembly 100 adapted for left heart support, in accordance with aspects of the disclosure. As shown in FIG. 1, an intracardiac blood pump assembly adapted for left heart support may include an elongate catheter 102, a motor 104, a cannula 110, a blood inflow cage 114 arranged at or near the distal end 112 of the cannula 110, a blood outflow cage 106 arranged at or near the proximal end 108 of the cannula 110, and an optional atraumatic extension 116 arranged at the distal end of the blood inflow cage 114.

In some aspects of the technology, motor 104 may be configured to rotatably drive an impeller (not shown), thereby generating suction sufficient to draw blood into cannula 110 through the blood inflow cage 114, and to expel the blood out of cannula 110 through the blood outflow cage 106. In that regard, the impeller may be positioned distal of the blood outflow cage 106, for example, within the proximal end 108 of the cannula 110 or within a pump housing 107 coupled to the proximal end 108 of the cannula 110. In some aspects of the technology, rather than the impeller being driven by an onboard motor 104, the impeller may instead be coupled to an elongate drive shaft (or drive cable) which is driven by a motor located external to the patient.

Catheter 102 may house electrical lines coupling the motor 104 to one or more electrical controllers and/or sensors. Alternatively, where the impeller is driven by an external motor, an elongate drive shaft may pass through catheter 102. Catheter 102 may also include a purge fluid conduit, a lumen configured to receive a guidewire, etc.

The blood inflow cage 114 may include one or more apertures or openings configured to allow blood to be drawn into cannula 110 when the motor 104 is operating. Likewise, blood outflow cage 106 may include one or more apertures or openings configured to allow blood to flow from the cannula 110 out of the intracardiac blood pump assembly 100. Blood inflow cage 114 and outflow cage 106 may be composed of any suitable bio-compatible material(s). For example, blood inflow cage 114 and/or blood outflow cage 106 may be formed out of bio-compatible metals such as stainless steel, titanium, or biocompatible polymers such as polyurethane. In addition, the surfaces of blood inflow cage 114 and/or blood outflow cage 106 may be treated in various ways, including, but not limited to etching, texturing, or coating or plating with another material. For example, the surfaces of blood inflow cage 114 and/or blood outflow cage 106 may be laser textured.

Cannula 110 may include a flexible hose portion. For example, cannula 110 may be composed, at least in part, of a polyurethane material. In addition, cannula 110 may include a shape-memory material. For example, cannula 110 may comprise a combination of a polyurethane material and one or more strands or coils of a shape-memory material such as Nitinol. Cannula 110 may be formed such that it includes one or more bends or curves in its relaxed state, or it may be configured to be straight in its relaxed state. In that regard, as shown in the exemplary arrangement of FIG. 1, the cannula 110 may have a single pre-formed anatomical bend 118 based on the portion of the left heart in which it is intended to operate. Despite this bend 118, the cannula 110 may nevertheless also be flexible, and may thus be capable of straightening (e.g., during insertion over a guidewire), or bending further (e.g., in a patient whose anatomy has tighter dimensions). Further in that regard, cannula 110 may include a shape-memory material configured to allow the cannula 110 to be a different shape (e.g., straight or mostly straight) at room temperatures, and to form bend 118 once the shape-memory material is exposed to the heat of a patient's body.

Atraumatic extension 116 may assist with stabilizing and positioning the intracardiac blood pump assembly 100 in the correct position in the patient's heart. Atraumatic extension 116 may be solid or tubular. If tubular, atraumatic extension 116 may be configured to allow a guidewire to be passed through it to further assist in the positioning of the intracardiac blood pump assembly 100. Atraumatic extension 116 may be any suitable size. For example, atraumatic extension 116 may have an outer diameter in the range of 4-8 Fr. Atraumatic extension 116 may be composed, at least in part, of a flexible material, and may be any suitable shape or configuration such as a straight configuration, a partially curved configuration, a pigtail-shaped configuration as shown in the example of FIG. 1, etc. Atraumatic extension 116 may also have sections with different stiffnesses. For example, atraumatic extension 116 may include a proximal section that is stiff enough to prevent it from buckling, thereby keeping the blood inflow cage 114 in the desired location, and a distal section that is softer and has a lower stiffness, thereby providing an atraumatic tip for contact with a wall of the patient's heart and to allow for guidewire loading. In such a case, the proximal and distal sections of the atraumatic extension 116 may be composed of different materials, or may be composed of the same material with the proximal and distal sections being treated to provide different stiffnesses.

Notwithstanding the foregoing, as mentioned above, atraumatic extension 116 is an optional structure. In that regard, the present technology may also be used with intracardiac blood pump assemblies and other intracardiac devices that include extensions of different types, shapes, materials, and qualities. Likewise, the present technology may be used with intracardiac blood pump assemblies and other intracardiac devices that have no distal extensions of any kind.

As described herein, the intracardiac blood pump assembly 100 may be inserted percutaneously. For example, when used for left heart support, intracardiac blood pump assembly 100 may be inserted via a catheterization procedure through the femoral artery or axillary artery, into the aorta, across the aortic valve, and into the left ventricle. Once positioned in this way, the intracardiac blood pump assembly 100 may deliver blood from the blood inflow cage 114, which sits inside the left ventricle, through cannula 110, to the blood outflow cage 106, which sits inside the ascending aorta. In some aspects of the technology, intracardiac blood pump assembly 100 may be configured such that bend 118 will rest against a predetermined portion of the patient's heart when the intracardiac blood pump assembly 100 is in a desired location. Likewise, the atraumatic extension 116 may be configured such that it rests against a different predetermined portion of the patient's heart when the intracardiac blood pump assembly 100 is in the desired location.

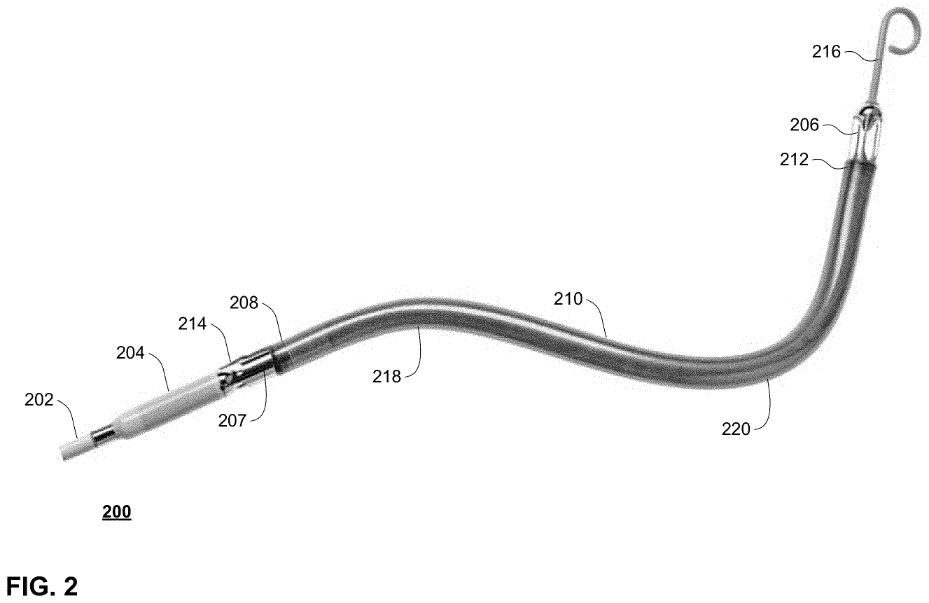

FIG. 2 depicts an exemplary intracardiac blood pump assembly 200 adapted for right heart support, in accordance with aspects of the disclosure. As shown in FIG. 2, an intracardiac blood pump assembly adapted for right heart support may include an elongate catheter 202, a motor 204, a cannula 210, a blood inflow cage 214 arranged at or near the proximal end 208 of the cannula 210, a blood outflow cage 206 arranged at or near the distal end 212 of the cannula 210, and an optional atraumatic extension 216 arranged at the distal end of the blood outflow cage 206.

As with the exemplary assembly of FIG. 1, motor 204 may be configured to rotatably drive an impeller (not shown), thereby generating suction sufficient to draw blood into cannula 210 through the blood inflow cage 214, and to expel the blood out of cannula 210 through the blood outflow cage 206. In that regard, the impeller may be positioned distal of the blood inflow cage 214, for example, within the proximal end 208 of the cannula 210 or within a pump housing 207 coupled to the proximal end 208 of the cannula 210. Here as well, in some aspects of the technology, rather than the impeller being driven by an onboard motor 204, the impeller may instead be coupled to an elongate drive shaft (or drive cable) which is driven by a motor located external to the patient.

The cannula 210 of FIG. 2 may serve the same purpose, and may have the same properties and features described above with respect to cannula 110 of FIG. 1. However, as shown in the exemplary arrangement of FIG. 2, the cannula 210 may have two pre-formed anatomical bends 218 and 220 based on the portion of the right heart in which it is intended to operate. Here again, despite the existence of bends 218 and 220, the cannula 210 may nevertheless also be flexible, and may thus be capable of straightening (e.g., during insertion over a guidewire), or bending further (e.g., in a patient whose anatomy has tighter dimensions). Further in that regard, cannula 210 may include a shape-memory material configured to allow the cannula 210 to be a different shape (e.g., straight or mostly straight) at room temperatures, and to form bends 218 and/or 220 once the shape-memory material is exposed to the heat of a patient's body.

The catheter 202 and atraumatic extension 216 of FIG. 2 may serve the same purpose and may have the same properties and features described above with respect to catheter 102 and atraumatic extension 116 of FIG. 1. Likewise, other than being located at opposite ends of the cannula from those of FIG. 1, the blood inflow cage 214 and blood outflow cage 206 of FIG. 2 may be similar to the blood inflow cage 114 and blood outflow cage 106 of FIG. 1, and thus may have the same properties and features described above.

Like the exemplary assembly of FIG. 1, the intracardiac blood pump assembly 200 of FIG. 2 may also be inserted percutaneously. For example, when used for right heart support, intracardiac blood pump assembly 200 may be inserted via a catheterization procedure through the femoral vein, into the inferior vena cava, through the right atrium, across the tricuspid valve, into the right ventricle, through the pulmonary valve, and into the pulmonary artery. Once positioned in this way, the intracardiac blood pump assembly 200 may deliver blood from the blood inflow cage 214, which sits inside the inferior vena cava, through cannula 210, to the blood outflow cage 206, which sits inside the pulmonary artery.

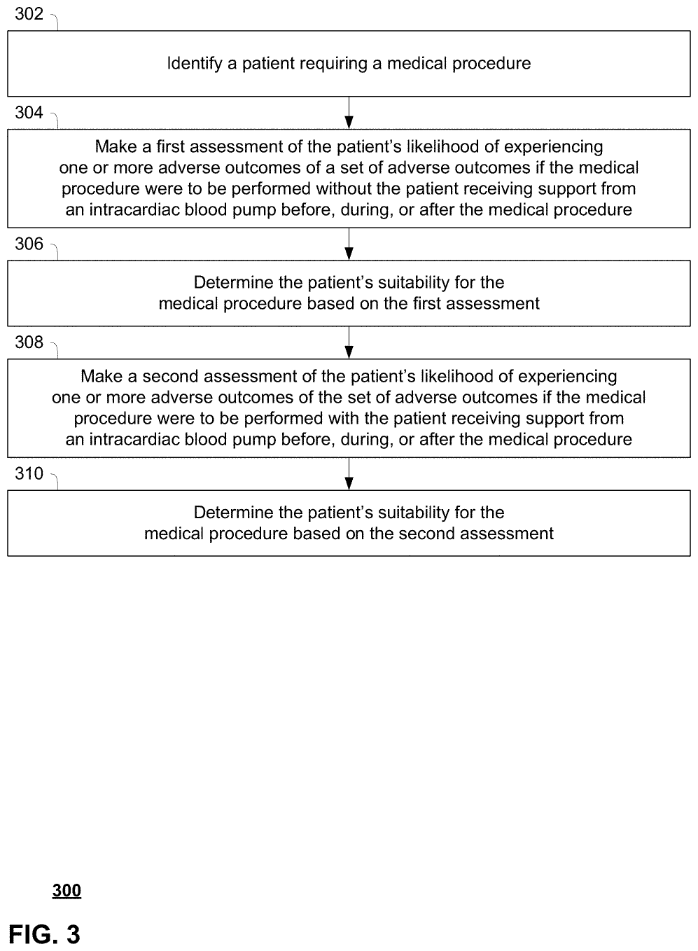

FIG. 3 depicts an exemplary method 300 for assessing whether a patient may benefit from treatment with an intracardiac blood pump (e.g., intracardiac blood pump assembly 100 or 200) in association with a high-risk medical procedure.

In that regard, in step 302, a patient is identified as requiring a medical procedure. As described herein, this medical procedure may include a cardiac procedure such as a mitral valve repair, mitral valve replacement, ventricular tachycardia ablation, or atrial fibrillation ablation. In some instances, such a cardiac procedure may be performed after an emergent cardiac procedure has been performed (e.g., after a patient has been treated for an emergent cardiac event). The medical procedure also may be any type of surgery or other procedure directed to an area of the body other than the heart or the “great vessels” that deliver blood to and from the heart. For example, the procedure may include a laparoscopic procedure, such as a laparoscopic bariatric procedure.

Next, an assessment may be made to determine the patient's risk to undergoing such a medical procedure. For example, as shown in step 304, a first assessment may be made to determine the patient's likelihood of experiencing one or more adverse outcomes of a set of adverse outcomes if the medical procedure were to be performed without the patient receiving support from an intracardiac blood pump before, during, or after the medical procedure. In some aspects of the technology, the set of adverse outcomes may include any potential adverse outcome known to be correlated with the medical procedure, such as hypotension (e.g., as may be caused by anesthesia), pulmonary edema, ventricular fibrillation, exacerbated ischemia, myocardial ischemia, hemodynamic instability and/or collapse, cardiac arrest, death, etc. Likewise, in some aspects of the technology, the patient's likelihood of experiencing one or more adverse outcomes of the set of adverse outcomes may be based on any suitable criterion or criteria, including, but not limited to: relevant information about the patient, such as the patient's age, height, weight, body mass index, blood pressure, cholesterol levels, liver function, kidney function, existing medical conditions (e.g., diabetes, autoimmune disorders, heart disease), personal medical history, family medical history; statistics regarding the rates of each adverse outcome in the population generally; and statistics regarding the rates of each adverse outcome in patients sharing one or more traits with the patient.

In step 306, the patient's suitability for the medical procedure is determined based on the first assessment. In some instances, the patient's suitability for the medical procedure may be determined based solely on the risks identified in the first assessment. The suitability also may be based on a balancing of those risks with one or more other risks, such as the patient's likelihood of experiencing one or more adverse outcomes if the medical procedure is not provided. For example, a patient who is assessed as having a high risk of heart failure during an elective cosmetic surgery may be deemed not suitable for that medical procedure. On the other hand, a patient who is assessed as having a high risk of heart failure during surgery to remove a cancerous tumor may be deemed suitable for that medical procedure if the patient is assessed to have an even higher risk of dying imminently from cancer if the tumor is not removed.

As described herein, a patient deemed unsuitable for a given medical procedure based on a risk of experiencing one or more adverse outcomes of a set of adverse outcomes (as discussed above with respect to steps 304 and 306) may still be eligible to have the procedure if the identified risk(s) may be mitigated by use of an intracardiac blood pump before, during, and/or after the procedure. In such cases, the patient's risk profile may be assessed a second time using the assumption that such support is provided, and the patient's suitability for the medial procedure may be thereafter reconsidered. In the example of FIG. 3, it is assumed that such a second assessment is made.

Thus, in step 308, a second assessment may be made of the patient's likelihood of experiencing one or more adverse outcomes of the set of adverse outcomes as a result of the medical procedure if an intracardiac blood pump were to be used to support the patient's heart before, during, and/or after the medical procedure. Here as well, the set of adverse outcomes may include any adverse outcomes on which the first assessment is based. Further in that regard, the set of adverse outcomes may also include any adverse outcomes known to be correlated with the use of an intracardiac heart pump. Likewise, the patient's likelihood of experiencing one or more of the set of adverse outcomes may be based on the same criterion or criteria on which the first assessment was based, and may further reflect how the use of the intracardiac blood pump may change the patient's chances of experiencing each adverse outcome of the set of adverse outcomes.

Thus, for example, if the patient was deemed in the first assessment to have a risk of severe hypotension while under anesthesia based on one or more criteria (e.g., age and/or prior medical conditions), the patient's second assessment may reflect a lower risk of hypotension based on the use of the intracardiac blood pump while the patient is under anesthesia and/or while the patient is recovering from the procedure. Likewise, if the patient was deemed in the first assessment to have a risk of hemodynamic collapse based on one or more criteria (e.g., inability to withstand the stress of the medical procedure due to obesity, diabetes, heart disease, etc.), the patient's second assessment may reflect a lower risk of hemodynamic collapse based on the use of an intracardiac blood pump to reduce the load on the patient's heart before, during, and/or after the procedure.

Finally, in step 310, the patient's suitability for the medical procedure may be determined based on the second assessment. Here as well, the patient's suitability for the medical procedure may be determined based solely on the risks identified in the second assessment. Likewise, in some aspects of the technology, the patient's suitability also may be based on a balancing of those risks with one or more other risks such as the patient's likelihood of experiencing one or more adverse outcomes if the medical procedure were not provided.

As will be appreciated, in some instances, although a patient may be deemed not suitable for a given medical procedure based on the first assessment, the patient may be deemed suitable for that procedure based on the second assessment so long as an intracardiac blood pump is used to support the patient's heart before, during, and/or after the procedure.

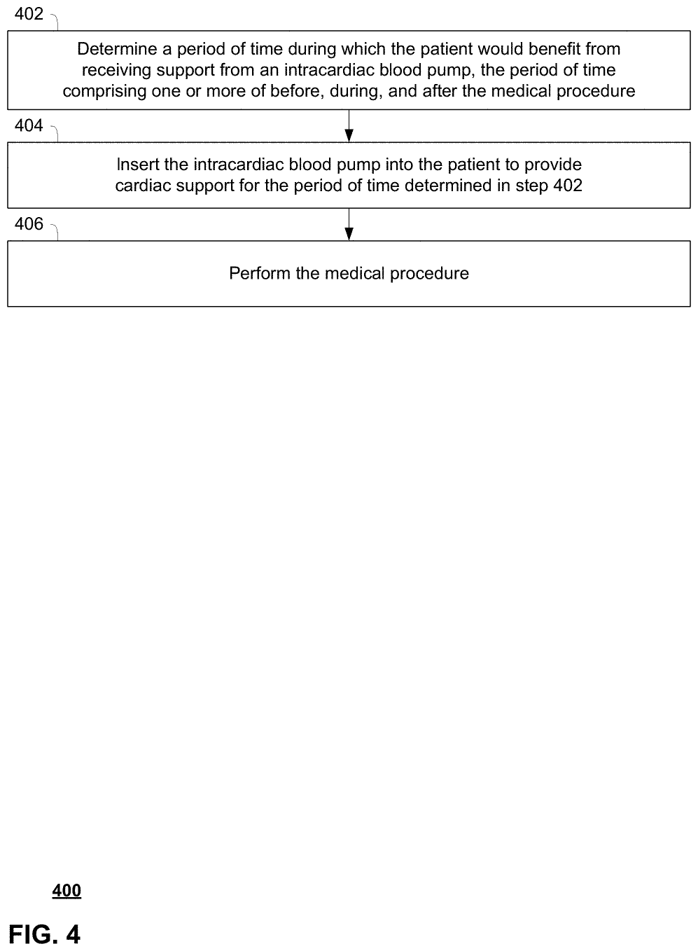

FIG. 4 depicts an exemplary method 400 for treating a patient using an intracardiac blood pump (e.g., intracardiac blood pump assembly 100 or 200) in association with a medical procedure. In that regard, method 400 may be employed if, according to method 300, it has been determined that a patient would benefit from the use of an intracardiac blood pump assembly during a medical procedure that would otherwise be considered high-risk for the patient, and/or in instances in which the patient would be unsuitable for the medical procedure without support from an intracardiac blood pump assembly (see steps 306 and 310 of FIG. 3).

In step 402, a determination may be made regarding a period of time during which the patient would benefit from receiving support from the intracardiac blood pump. The period of time may be one or more of before, during, and after the medical procedure. In that regard, an intracardiac blood pump may be used in a variety of ways to lower and/or eliminate risks of the patient experiencing a cardiac event during or after the medical procedure. For example, an intracardiac blood pump may be used before the medical procedure to allow the heart to rest prior to the medical procedure, thus potentially lowering the risk that the heart will subsequently be overcome by the trauma of the medical procedure. Likewise, an intracardiac blood pump may be used during the medical procedure to lower the load on the heart and maintain blood flow through the body, thus potentially lowering the risk of ventricular fibrillation, exacerbated ischemia, myocardial ischemia, pulmonary edema, hemodynamic collapse, cardiac arrest, death, etc., which may occur during the medical procedure. Further, an intracardiac blood pump may be used after the medical procedure to allow the heart to recover, and thus lessen the risk of post-operative cardiac events such as heart attack, ventricular fibrillation, exacerbated ischemia, myocardial ischemia, pulmonary edema, hemodynamic collapse, cardiac arrest, death, etc. Depending on the situation, the intracardiac blood pump may thus be used: (a) only before the procedure; (b) before and during the procedure; (c) before, during, and after the procedure; (d) only before and after the procedure, but not during the procedure; (e) only during the procedure; (f) only during and after the procedure; or (g) only after the procedure.

In step 404, the intracardiac blood pump may be inserted into the patient to provide cardiac support for the period of time determined in step 402. Any suitable way of inserting, positioning, and providing cardiac support using the intracardiac blood pump may be used in this regard, including the methods of providing left-heart and right-heart support that are described above with respect to FIGS. 1 and 2, respectively.

In step 406, the medical procedure may be performed. In some aspects of the technology, steps 404 and 406 may take place simultaneously or their order may be reversed from what is shown in exemplary method 400. For example, in cases where it is determined in step 402 that the intracardiac blood pump is only to be used after the medical procedure, the medical procedure (step 406) may take place before insertion of the intracardiac blood pump (step 404). Likewise, in cases where the intracardiac blood pump is to be used during the medical procedure, the intracardiac blood pump may nevertheless be inserted into the patient (step 404) at some point after the medical procedure (step 406) has begun. In some aspects of the technology, the step of performing the medical procedure may include or commence with placing the patient under anesthesia.

As will be appreciated, the exemplary methods 300 and 400 may be used to identify and treat patients who do not have an identified heart condition, but who nevertheless may be at risk for a cardiac event during a medical procedure, and thus would benefit from receiving support from an intracardiac blood pump before, during, and/or after the procedure. For example, an elderly patient may be in good health, with a healthy heart. However, based on age, required medications, medical history, or other factors, that patient may be deemed not suitable for a medical procedure (e.g., an arthroplasty procedure such as a knee or hip replacement) based on a risk that the patient will experience an adverse outcome such as hypotension during anesthesia, which may in turn cause a stroke, heart attack, acute kidney injury, postoperative neurological declines, and/or increased postoperative mortality rates. Similarly, the same patient may be deemed not suitable for the medical procedure based on a risk that the stress of the procedure may bring on hemodynamic collapse despite the patient having a healthy heart under normal circumstances. Where the gravity of the risks posed by the medical procedure outweigh the potential benefits of the procedure (e.g., the patient having a repaired knee or hip), the patient may be denied treatment. However, if those risks may be lowered or eliminated by supporting the patient's heart with an intracardiac blood pump, the patient may be able to safely receive a medical procedure that meaningfully extends and/or improves the quality of his or her life.

The exemplary methods 300 and 400 also may be used to identify and treat patients who have one or more heart conditions that create a risk of a cardiac event during a medical procedure (cardiac or noncardiac procedure), and thus would benefit from receiving support from an intracardiac blood pump before, during, and/or after the procedure. For example, a patient may be obese, and may be suffering from various associated medical conditions such as diabetes, high blood pressure, dyslipidemia, high C-reactive protein levels, fatty liver, etc. In addition, the patient may also have one or more heart conditions (e.g., coronary heart disease, NYHA class I, II, III, or IV heart failure, etc.) that could complicate the medical procedure, but nevertheless will not be addressed with an immediate cardiac procedure to address those heart conditions (e.g., angioplasty or stent placement). For example, the patient's heart conditions may not yet be severe enough to warrant such a cardiac procedure, may not be of a type that can be addressed with a cardiac procedure, or may be severe enough to warrant an eventual cardiac procedure but yet not be severe enough to take precedence over the present medical procedure. Such a patient, for example, may benefit from weight loss, and thus may be a prime candidate for bariatric surgery under normal circumstances. However, the patient may nevertheless be turned down for such a procedure because the patient's heart conditions and/or other medical issues place the patient at an unacceptably high risk of experiencing a cardiac event during the bariatric surgery. Here as well, an intracardiac blood pump may be used to lower and/or eliminate some or all of these risks by supporting the heart before the procedure (e.g., to allow the heart to rest prior to the trauma of the surgery), during the procedure (e.g., to reduce load on the heart, maintain blood flow, and thus lessen the risk during the surgery of ventricular fibrillation, exacerbated ischemia, myocardial ischemia, pulmonary edema, hemodynamic collapse, cardiac arrest, etc.), and/or after the procedure (e.g., to allow the heart to recover, and thus lessen the risk of post-operative cardiac events). In this way, the intracardiac heart pump may allow the patient to receive a life-saving medical procedure that might otherwise be unavailable.

From the foregoing and with reference to the various figures, those skilled in the art will appreciate that certain modifications can also be made to the present disclosure without departing from the scope of the same. While several aspects of the disclosure have been shown in the figures, it is not intended that the disclosure be limited thereto, as it is intended that the disclosure be as broad in scope as the art will allow and that the specification be read likewise. Therefore, the above description should not be construed as limiting, but merely as exemplifications of particular aspects of the present technology.

1. A method of administering medical treatment, comprising:
identifying a patient requiring a medical procedure;
making a first assessment of the patient's likelihood of experiencing one or more adverse outcomes of a set of adverse outcomes if the medical procedure were to be performed without the patient receiving support from an intracardiac blood pump before, during, or after the medical procedure;
determining the patient's suitability for the medical procedure based on the first assessment;
making a second assessment of the patient's likelihood of experiencing one or more adverse outcomes of the set of adverse outcomes if the medical procedure were to be performed with the patient receiving support from an intracardiac blood pump at least before, during, or after the medical procedure;
determining the patient's suitability for the medical procedure based on the second assessment; and
inserting the intracardiac blood pump into the patient to provide cardiac support at least before, during, or after the medical procedure.

2. The method of claim 1, further comprising determining a period of time during which the patient would benefit from receiving support from the intracardiac blood pump, wherein the determined period of time comprises one or more of before, during, or after the medical procedure.

3. The method of claim 2, wherein inserting the intracardiac blood pump into the patient to provide cardiac support is performed for the determined period of time.

4. The method of claim 1, further comprising performing the medical procedure on the patient.

5. The method of claim 4, wherein inserting the intracardiac blood pump into the patient is performed before, at the same time as, or after performing the medical procedure.

6. The method of claim 1, wherein the medical procedure includes a noncardiac medical procedure.

7. The method of claim 1, wherein the intracardiac blood pump is configured to provide left heart support.

8. The method of claim 1, wherein the intracardiac blood pump is configured to provide right heart support.

9. The method of claim 1, wherein the medical procedure requires the patient to be anesthetized.

10. The method of claim 1, wherein the medical procedure includes one or more of laparoscopic surgery, tumor resection, or gastrointestinal surgery.

11. The method of claim 1, wherein the medical procedure includes one or more of mitral valve repair, mitral valve replacement, ventricular tachycardia ablation, or atrial fibrillation ablation.

12. The method of claim 1, wherein the medical procedure includes knee or hip arthroplasty.

13. The method of claim 1, wherein the set of adverse outcomes includes one or more of hypotension, pulmonary edema, ventricular fibrillation, exacerbated ischemia, myocardial ischemia, hemodynamic collapse, cardiac arrest, stroke, heart attack, acute kidney injury, neurological decline, or death.

14. The method of claim 1, wherein the first assessment or the second assessment is based on one or more of the patient's age, height, weight, body mass index, blood pressure, cholesterol levels, liver function, kidney function, existing medical conditions, personal medical history, or family medical history.

15. The method of claim 1, wherein the first assessment or the second assessment is based on whether the patient has one or more of diabetes, an autoimmune disorder, or heart disease.

16. The method of claim 1, wherein the first assessment or the second assessment is based on statistics regarding how prevalent each adverse outcome in the set of adverse outcomes is in a given population.

17. The method of claim 16, wherein the given population comprises a group of people sharing one or more traits with the patient.

18. The method of claim 1, wherein the second assessment is based on a likelihood of the patient experiencing one or more adverse outcomes of the set of adverse outcomes as a result of implantation of the intracardiac blood pump in the patient.
